(19) United States
(12) Patent Application Publication
(10) Pub. No.: US 2003/0171408 A1
(43) Pub. Date: Sep. 11, 2003

(54) Therapeutic method of delivering a medicament to avoid irritating effects on membranes of user

(76) Inventor: Jay L. Caplan, Brentwood, TN (US)

Correspondence Address: Thomas A. Beck, 26 Rockledge Lane, New Milford, CT, 06776, US

(21) Appl. No.: 10/095,287

(22) Filed: Mar. 11, 2002

(51) Int. Cl. A61K 31/4439

(52) U.S. Cl. 514/343

(57) ABSTRACT

A composition for administration to the nasal mucosa or as an oral inhalant comprises a molecule which is the reaction product of nicotine with two molecules of a sweetener such as acesulfamic acid. The resultant molecule of nicotine diacesulfamate in a pharmaceutically acceptable solvent offers an improved side effect profile as regards irritation and unpleasant taste for the subject using the composition.

### FIELD OF THE INVENTION

The invention relates to an improved therapeutic method of delivering a medicament into a life system, and more particularly, the invention comprises the new use of the reaction product of one or more materials characterized as “sweeteners” and a compound such as a pharmaceutical, a synthetic non-alkaloidal medical organic base, or a medicament such as nicotine, any of which, when inhaled orally or nasally, produces side effects including burning, watering and other unpleasant feelings in the throat, nose, lungs, eyes, and other mucous membranes of the user.

### BACKGROUND OF THE INVENTION

For the purpose of explication of the present invention, reference is made primarily to the medicament embodiment which is nicotine, the major alkaloid of tobacco. Nicotine is a simple pyridine which bears a reduced pyrrole (pyrrolidine) unit as a substituent in the β position. It is convenient to illustrate the present invention using the nicotine embodiment since the molecule has two reactive sites through which one can modify the irritating properties which nicotine possesses. Nonetheless, the exemplification of nicotine herein should not be construed as limiting the scope of the invention.

In industrialized countries about one fourth to one third of the adult population smokes cigarettes, resulting in a major avoidable cause of morbidity and mortality. Smoking is a contributory or causative factor in a number of diseases including respiratory diseases such as emphysema, chronic bronchitis, lung infections, and lung cancer; cardiovascular disease; gastric and duodenal ulcers; and cancer of the lung, oral cavity, larynx and esophagus.

Most regular smokers become addicted to, or dependent upon, the pharmacological effects of nicotine in tobacco smoke. Nicotine is rapidly absorbed across the blood brain barrier and exerts a direct action on nicotine receptors in the spinal cord, autonomic ganglia and adrenal medulla. For more detailed information on the pharmacological effects of nicotine see, for example, Oates and Wood, New Eng. J. Med. 319:1318, 1988. Nicotine itself has been implicated as a contributory factor in coronary heart diseases, peripheral vascular disease and hypertension.

Although nicotine is responsible for the addictive nature of cigarette smoking, most of the harmful health effects of smoking are attributable to other constituents in cigarette smoke (The Lancet, 337:1191, May 18, 1991). The combustion of tobacco in cigarettes results in the production of up to 4,000 compounds and the inhalation of such unwanted by-products as tar, combustion gases and a range of carcinogens. Nicotine may be nitrosated to form highly carcinogenic tobacco-specific N-nitrosamines in tobacco smoke, or in the cured smokeless tobacco for use as chewing tobacco or snuff. It is an unfortunate feature of cigarette smoking that the negative consequences of nicotine addiction are largely manifested by the inhalation of toxic and carcinogenic materials generated by the combustion of tobacco.

Addiction to smoking is based upon a pharmacological dependence on nicotine, an addiction comparable to that arising from the use of heroin. There are a number of acute symptoms of smoking cessation relating to nicotine withdrawal including irritability, anxiety, insomnia and a craving for nicotine. The addictive nature of nicotine poses a major obstacle to those who wish to quit smoking, and a number of approaches have been developed to aid individuals in their efforts to stop smoking. The more successful of these involve therapy with nicotine substitutes such as chewing gum, nicotine patches, nicotine nasal sprays, nicotine inhalers and the like. However, as discussed in more detail below, these approaches have met with limited user acceptance and limited success. In addition, there are individuals who are unable to stop despite repeated attempts because of the addictive nature of nicotine. These individuals could benefit from a product which fulfilled their craving for nicotine but did not have the same detrimental health consequences as cigarettes.

Smoking is a uniquely effective form of systemic drug administration. As nicotine enters the circulation via the pulmonary circulation, it is speedily transported to the brain. Smokers achieve a rapid peak in nicotine levels in the blood within one or two minutes after finishing a cigarette. Nicotine substitutes generally contain nicotine in solid form, in a vapor or in solution. Since nicotine is a base, these preparations are alkaline.

With respect to nicotine gum, it is known that nicotine, even at an alkaline pH, is absorbed rather slowly across the mucous membranes of the oral cavity, so absorption by this route does not produce the very rapid increase in nicotine levels associated with cigarette smoking. Therefore, buccal absorption has proved to have limited use in simulating the effects of cigarette smoking and lessening the adverse symptoms of nicotine withdrawal. Lower nicotine levels are achieved from chewing nicotine gum as compared to smoking cigarettes, and the gum has been associated with gastrointestinal side effects, hiccups, mouth ulcers and sore throat. The amount of nicotine absorbed also is highly variable and is dependent upon the chewing and swallowing actions of the user over a prolonged period of time.

Nicotine patches are associated with skin irritation at the application site. Both nicotine gum and dermal patches result in slow absorption of nicotine, not frequently effective in satisfying the patient's craving for cigarettes. This may be one of the reasons for the lack of success of these forms of therapy in weaning subjects from smoking.

Inhalers of nicotine are in current use and involve the user inhaling volatile products or mists including nicotine into the mouth from the inhaler. These products are for the replacement of smoking or tobacco cessation purposes. This usage causes side effects including burning, watering and other unpleasant feelings experiences in the throat, nose, lungs, eyes, and other mucous membranes of the user.

Self-propelled inhalers which contain nicotine in solution have also been used as cigarette substitutes. An example is the self-propelled inhaler formulation of Jacobs in U.S. Pat No. 4,635,651 (“the '651 patent”). Such formulations are packaged in pressurized metered dose delivery systems. As shown in the '651 patent, these delivery systems contain a water based aerosol formulation and a propellant such as pressurized freon which are stored in a pressurized storage container. When the device is used by an individual, the user aims the delivery system into their mouth. The user then inhales while causing a pre-metered dose of aerosol to be forced from the storage container and expelled at high speed into the user's mouth.

It is well established that nicotine is easily absorbed nasally. Nicotine concentrations in the blood of regular users of dry snuff are similar to those of cigarette smokers and peak concentrations after a single pinch of snuff is reached in a time similar to that for smoking a cigarette. The absolute bioavailability of nicotine applied to different nasal regions has been measured by Johansson et al., Eur. J. Clin. Pharmacol. 41,585 (1991) in man. Single doses of one mg were given and plasma concentrations followed over 6 hours. Bioavailability, as compared to intravenous infusion (IV) was 60 to 75%. The rate of absorption was fast in as much as, the maximum concentration was reached within about 10 minutes. No differences could be found for different nasal treatments.

Nasal sprays containing nicotine have been suggested as an alternative approach for smoking cessation. The prior art has described various devices for the better delivery of nicotine. For example, WO 87/038,13 a spray device with an electronic timer restricting doses to a predetermined number per session is described.

A nasal aerosol spray supplying nicotine for anti-smoking therapy is mentioned as an advantage. In U.S. Pat. No. 4,655,231 an improved snuff for nasal application of nicotine containing a pure nicotine salt, a water soluble diluent and coloring and flavoring is described. The water soluble diluent is preferably an organic acid. The mixture allows rapid application of nicotine.

Other methods and means for supplying nicotine for anti-smoking therapy are found in U.S. Pat. No. 5,656,255 to Jones and U.S. Pat. 5,721,257 to Baker, et al., the contents of which are hereby incorporated by reference herein. The nasal nicotine systems discussed above were designed to give rapid absorption of nicotine, followed by a rapid decrease in the level of absorbed nicotine, mimicking the effect of smoking a cigarette. More recently, Sutherland et al. Lancet 340:324 (1992), suggested that the rapid absorption of the nicotine when given nasally may be an important factor for smokers for whom other forms of replacements are too slow.

A major drawback in the use of nasal nicotine systems is the burning, watering and other unpleasant feelings experiences in the throat, nose, lungs, eyes, and other mucous membranes of the user.

In addition to serving as a means to stop smoking, the application of a nicotine solution pursuant to the process of the present invention also can reduce symptoms of a medical condition in a person who has a medical condition. The nicotine is administered in a therapeutically effective amount to achieve a sufficient blood level of the nicotine to reduce the symptoms. the other medical conditions beside smoking tobacco which are treated by nicotine include, but are not limited to, addiction to chewing tobacco, attention deficit disorder, Alzheimer's disease, Parkinson's disease, inability to regulate body weight at a level proper for body height, depression, ulcerative colitis, and combinations thereof.

### SUMMARY OF THE INVENTION

The invention provides an improved means of delivering a medicament into a life system by means of a nasal delivery article or by an oral inhaler. In the case of irritating pharmaceuticals and nicotine, etc., the improvement is the substantially reduced sensation of burning, watering and other unpleasant feelings experienced in the throat, nose, lungs, eyes, and other mucous membranes of the user.

The object of the invention is to allow the irritation-free delivery of a pharmaceutical, a synthetic non-alkaloidal medical organic base, or a medicament such as, for example, nicotine, via oral or nasal means, the identical use of which, heretofore, would have irritated the mucosa of the user. The use of the modified compound in accordance with the present invention results in obtaining the beneficial effect of the compound without irritating the user's mucosa, also referred to as the mucous membrane, which includes, but is not limited to the alveolar, lingual, masticatory, nasal and oral membranes.

In the case of nicotine, the delivery nasally or by oral inhaler is improved by reacting nicotine with acesulfamic acid. The resultant compound, or complex is, for example, nicotine mono-or di-acesulfamate. The aforementioned reaction product substantially reduces the unpleasant sensory properties of the prior art systems and is much more pleasant for the individual to use. It is absorbed well and delivers nicotine to the bloodstream in a therapeutic manner for the prevention (or alleviation) of smoking tobacco and chewing tobacco dependence as well as for other conditions which are treated by nicotine and include, but are not limited to, addiction to chewing tobacco, attention deficit disorder, Alzheimer's disease, Parkinson's disease, inability to regulate body weight at a level proper for body height, depression, ulcerative colitis, and combinations thereof.

The method of the present invention works equally effectively when a pharmaceutical, a synthetic non-alkaloidal medical organic base, or other medicament is reacted with the acesulfamate and inhaled orally or nasally.

### BRIEF DESCRIPTION OF THE DRAWINGS

FIG. 1 is the chemical molecular formula for nicotine diacesulfamate, the preferred embodiment of the active ingredient used in accordance with the present invention.

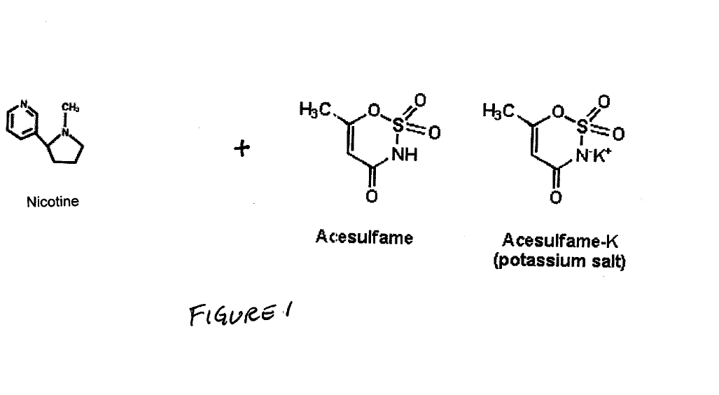

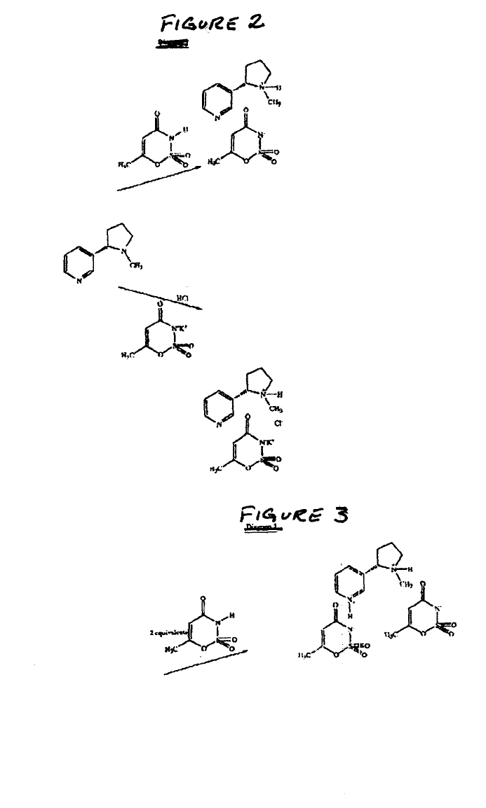

### DETAILED DESCRIPTION OF THE PREFERRED EMBODIMENT

The present invention relates to a method of applying a medicament composition of matter via aspiration of a liquid or powder into the nasal mucosa or via an inhaler containing a liquid or a powder as an oral inhalant. The preferred embodiment of said medicament comprises the reaction product of a molecule of nicotine and one or more molecules of acesulfamic acid, alone or in combination with another sweetener type compound in a suitable solvent or powder carrier.

The new use of the preferred medicament composition noted above involves using a product, and applying it using a nicotine nasal spray, for example, dispensed from a nasal spray, a nicotine inhaler, the typical plastic cylindrical tube containing active ingredient in a cartridge contained therein, or other similar suitable means.

As noted above, the present invention is an advance over the prior art in which unmodified nicotine was inhaled orally, or alternatively, the unmodified nicotine solution was delivered nasally. These means of delivery were both unsatisfactory due to a burning taste when inhaled orally or because of the irritation that the unmodified nicotine causes to the linings of the mucosa, particularly the nasal passages, throat, bronchial passages and lungs.

The resultant reaction product of nicotine diacesulfamate in a pharmaceutically acceptable solvent offers an improved side effect profile as regards nasal irritation of the subject using this medicament.

The improved property of non-irritation to the nasal lining is a compelling feature over the prior art which, more particularly, discloses an unmodified nicotine nasal spray in some aerosol form contained in a spray pump. The nicotine is delivered to the user by spraying it into the nostrils, and is rapidly absorbed through the nasal lining-membranes inside the nose into the bloodstream. Because of the rapid absorption of the nicotine in the nasal spray, the nicotine is delivered much more quickly into the human system than other nicotine replacement therapies. As noted above, the compelling contraindication to the use of the prior art spray is that the unmodified nicotine causes substantial nose and throat irritation. Other side effects from the nasal spray are watering eyes, sneezing and cough. It has been determined that the irritation of the nasal membranes and other areas is due to the use of unmodified nicotine.

The present invention uses acesulfame, which is 6-Methyl-1,2,3-Oxathiazin-4(3H)1,2,2-dioxide H [C₄H₄NO₄SH] in combination with the nicotine. The preferred acesulfame form is acesulfamic acid (acesulfame H) which is soluble in alcohol and non-polar solvents where it can react with the nicotine base. Acesulfamic acid is only sparingly soluble in water. However, the potassium salt, acesulfame K, may be used in acidic aqueous solution (such as HCl) along with the nicotine base. In this case, the nicotine diacesulfame forms along with KCl all in solution.

Although the alternate method of forming the preferred compound comprises reacting the acesulfamic salt (K) with nicotine in acidic aqueous solution, any suitable metal cation which renders the compound water soluble and is physiologically compatible with the user may be used.

One or more molecules of the acesulfame compound is/are reacted with nicotine to form a composition which when added to a suitable solvent and sprayed in the nostrils in a concentration of up to 100 mg/ml, promptly results in a lessening of the craving associated with nicotine dependence.

The carrier solvent used in the present invention is water, a water miscible solvent such as an alcohol or a physiologically harmless acid, preferably hydrochloric acid or other suitable inorganic or organic acids. Other ingredients may be added to the formulations, including, but not limited to, buffers, preservatives, thickening agents, flavoring and coloring.

While the acesulfame-nicotine compound is preferred, other sweetener type moieties may be combined with the nicotine molecule to obtain similar results. Some examples of the molecules which can conveniently replace or be used in conjunction with acesulfame are other oxathiazinone sweeteners, alitame, aspartame, and aspartame like di- and tri-peptides, cyclamate and other sulfamate sweeteners, glycyrrhizin, neotame, saccharin, gluconic acid.

The process for preparing the compound used in accordance with the present invention involves reacting in a suitable solvent, nicotine with one or more of the sweetener compounds detailed above, which are identical or different, or with their physiologically acceptable salts in the presence of a physiologically acceptable acid and then isolating the reaction product formed. The nicotine and sweetener moieties are present in a molar ratios of 1:1, or 1:2, wherein the 1:2 molar sweetener ratio may include mixtures of different sweeteners of varying ratios between them.

The compounds noted above and the methods for making them are described in U.S. patent application 2001 0,029,959, the contents of which are hereby incorporated by reference herein.

In addition to the means of delivery of the medicament by nasal spray, the present invention also embodies using a dry powder for delivering a nicotine containing medicament in the form of salts and complexes via inhalation.

The powder form consists of a flowable mixture comprising a blend of active ingredient and carrier in the form of a composite material at conditions enabling formation of the nicotine medicament suitable for delivery to the nasal passages, throat, buccal area, alveoli and/or lower airways of a person. A process for delivering a nicotine-containing medicament in the form of salts and complexes is described in PCT application PCT/CA95/00562, the contents of which are hereby incorporated by reference herein. A carrier material which can be used to contain the complex with nicotine in the form of dry powder includes cyclodextrins, sugars, starches and other system compatible compounds.

While the present invention is directed preferably to delivering nicotine to the human system to reduce the craving of the nicotine-dependent user, the use of the active ingredient, whether it be nicotine or some other medicament or pharmaceutical in a nasal spray to effectuate the passage of the active ingredient into the human system is an effective way to quickly deliver a measured amount of the medicament to the individual and reduce unpleasant side effects.

### EXAMPLE

A subject who was an addicted smoker was provided a metered nasal spray delivering 0.4 mg of nicotine equivalent in form of nicotine diacesulfamate. The nicotine salt was dissolved in water without any other additive ingredients. The subject used the spray once in each nostril and reported a relief from desire to smoke within two minutes.

No report was made of any irritation to nasal mucosa, nor to the throat or lungs. The subject said the taste and feelings in the nostril was not unpleasant, but distinctive.

### EXAMPLE

A subject is administered nicotine base 0.5 mg per metered nasal spray of Nicotrol (Pharmacia, Sweden) unmodified nicotine in solution, and reports unpleasant burning sensation in the nostrils, nasal passages and in the throat. Additionally, watering of the eyes is noted.

### EXAMPLE

This Example shows applicability of the present invention to powder delivery of the active ingredients via microspheres containing starch as well as in aqueous solution as shown above.

A subject is administered nicotine by means of a powder nasal spray. The nicotine is prepared as the diacesulfamate and is encapsulated in starch microspheres. No burning feeling is noted on administration through the nasal spray ingestion. The subject using the spray reports relief from the cravings to smoke soon after use.

### EXAMPLE

A subject is administered nicotine by means of an oral inhaler. The nicotine is prepared as the diacesulfamate and is prepared in water solution. No burning feeling is noted on administration through the oral inhaler to neither the buccal area nor the throat or lungs. The subject using the inhaler reports relief from craving to smoke soon after use.

The present invention provides an improvement of kinetics of nasal over oral, skin or buccal delivery. Accordingly, while the preferred embodiment of the present invention is directed towards the use of the nicotine diacesulfamide composition with or without other sweeteners, the method of the present invention and the use of the acesulfamates/sweeteners disclosed herein can be successfully utilized for other disease treatments in addition to tobacco cessation treatment. Thus the instant invention can be conveniently used on other medicament and drug bases that result in bad irritation to the mucosa when incorporated by nasal spray or oral inhaler usage.

Thus, while there have been shown, described and pointed out fundamental novel features of the invention as applied to currently preferred embodiments thereof, it will be understood that various omissions and substitutions and changes in the form and details of the method and compositions illustrated, and in their operation, may be made by those skilled in the art without departing from the spirit of the invention. It is the intention, therefore, to be limited only as indicated by the scope of the claims appended herewith.

What I claim and desire to protect by Letters Patent is:

1. A method of providing an improved means of delivering a therapeutically effective dosage sufficient to provide the desired therapeutic result and to ameliorate any adverse effects on the nasal passages, throat, nose, lungs, eyes, and other mucous membranes of the user resulting from delivery therein by nasal or oral means to an individual, comprising applying the reaction product of a compound selected from the group consisting of a pharmaceutical, a synthetic non-alkaloidal medical organic base, or a medicament and a sweetener in a solvent to said individual.

2. The method defined in claim 1 wherein said compound is a medicament.

3. The method defined in claim 1 wherein said medicament is nicotine.

4. The method defined in claim 1 wherein said sweetener is acesulfame.

5. The method defined in claim 4 wherein said sweetener is acesulfame.

6. The method defined in claim 5 wherein said means of delivery is by nasal delivery.

7. The method defined in claim 6 wherein said sweetener is selected from the group consisting of acesulfame, other oxathiazinones, alitame, aspartame and aspartame like di- and tri peptides, cyclamate, sulfamate, glycyrrhizin, neotame, saccharin and gluconic acid.

8. The method defined in claim 8 wherein said sweetener is diacesulfamate.

9. The method defined in claim 9 wherein said solvent is selected from the group consisting of water, ethanol, isopropyl alcohol, other alcohols, propylene glycol, polyethylene glycol, vegetable oils, n-ethylene glycol(s), halogenated organic solvents, or povidone.

10. The method defined in claim 9 wherein said solvent is water.

11. The method defined in claim 6 wherein said composition has a concentration of between about 0.001 to 100.0 mg nicotine/ml by weight/volume of composition.

12. The method defined in claim 1 wherein said means of delivery is by means of oral inhaler.

13. The method defined in claim 4 wherein said sweetener is selected from the group consisting of acesulfame, other oxathiazinones, alitame, aspartame and aspartame like di- and tri peptides, cyclamate and other sulfamate sweeteners, glycyrrhizin, neotame, saccharin and gluconic acid.

14. The method defined in claim 13 wherein said sweetener is diacesulfamate.

15. The method defined in claim 14 wherein said solvent is selected from the group consisting of water, ethanol, isopropyl alcohol, other alcohols, propylene glycol, polyethylene glycol, vegetable oils, n-ethylene glycol, halogenated organic solvents, or povidone.

16. The method defined in claim 15 wherein said solvent is water.

17. The method defined in claim 16 wherein said composition has a concentration of between about 0.001 to 100.0 mg nicotine/ml by weight/volume of composition.

18. The method defined in claim 1 wherein said compound is a synthetic nonalkaloidal medical organic base.

19. The method defined in claim 18 wherein said organic base is ingested via an inhaler.

20. The method defined in claim 18 wherein said sweetener is acesulfame.

21. The method defined in claim 20 wherein said means of delivery is by nasal delivery.

22. The method defined in claim 21 wherein said sweetener is selected from the group consisting of acesulfame, other oxathiazinones, alitame, aspartame and aspartame like di- and tri peptides, cyclamate, sulfamate, glycyrrhizin, neotame, saccharin and gluconic acid.

23. The method defined in claim 22 wherein said sweetener is diacesulfamate.

24. The method defined in claim 23 wherein said solvent is selected from the group consisting of water, ethanol, isopropyl alcohol, other alcohols, propylene glycol, polyethylene glycol, vegetable oils, n-ethylene glycol, halogenated organic solvents, or povidone.

25. The method defined in claim 24 wherein said solvent is water.

26. The method defined in claim 25 wherein said composition has a concentration of between about 0.001 to 100.0 mg nicotine/ml by weight/volume of composition.

27. The method defined in claim 1 wherein said compound is a pharmaceutical.

28. The method defined in claim 27 wherein said sweetener is acesulfame.

29. The method defined in claim 28 wherein said means of delivery is by nasal delivery.

30. The method defined in claim 29 wherein said sweetener is selected from the group consisting of acesulfame, other oxathiazinones, alitame, aspartame and aspartame like di- and tri peptides, cyclamate, sulfamate, glycyrrhizin, neotame, saccharin and gluconic acid.

31. The method defined in claim 30 wherein said sweetener is diacesulfamate.

32. The method defined in claim 31 wherein said solvent is selected from the group consisting of water, ethanol, isopropyl alcohol, other alcohols, propylene glycol, polyethylene glycol, vegetable oils, n-ethylene glycol(s), halogenated organic solvents, or povidone.

33. The method defined in claim 32 wherein said solvent is water.

34. The method defined in claim 33 wherein said composition has a concentration of between about 0.001 to 100.0 mg nicotine/ml by weight/volume of composition.
